(19) United States
(12) Patent Application Publication
(10) Pub. No.: US 2020/0090800 A1
(43) Pub. Date: Mar. 19, 2020

(54) SYSTEMS AND METHODS FOR PREDICTING SKIN TREATMENT OUTCOMES USING SWEAT PH INFORMATION

(71) Applicant: L'Oreal, Paris (FR)

(72) Inventor: Rafal Michal Pielak, El Cerrito, CA (US)

(73) Assignee: L'Oreal, Paris (FR)

(21) Appl. No.: 16/572,360

(22) Filed: Sep. 16, 2019

Related U.S. Application Data
(60) Provisional application No. 62/732,497, filed on Sep. 17, 2018.

(51) Int. Cl. G16H 15/00; G16H 30/20; G16H 20/10

(52) U.S. Cl. CPC G16H 15/00; G16H 20/10; G16H 30/20

(57) ABSTRACT

A colorimetric sensor for pH measurement may be used (e.g. litmus paper, microfluidic device with chemistry for colorimetric pH measurement) to collect sweat pH information from a subject. An application is provided on a computing device that detects and analyzes the color change in order to determine the pH of sweat detected by the colorimetric sensor. Based on the determined sweat pH values and questionnaire responses, the application may recommend a skin regimen/treatment to restore skin function. The application may also provide visualizations of predicted effects of the skin regimen on a skin condition of the subject.

### CROSS-REFERENCE(S) TO RELATED APPLICATION(S)

This application claims the benefit of Provisional Application No. 62/732,497, filed Sep. 17, 2018, the entire disclosure of which is hereby incorporated by reference herein for all purposes.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a system for generating predictive visualizations of a result of a skin treatment regimen is provided. The system comprises a server computing system, at least one physical collection device, and a mobile computing device. The server computing system comprises one or more computing devices. The at least one physical collection device is configured to produce a color indicative of a sweat pH of a subject on whose skin the physical collection device is placed. The mobile computing device comprises at least one processor, a display device, a camera, and a non-transitory computer-readable medium having computer-executable instructions stored thereon. The instructions, in response to execution by the at least one processor, cause the mobile computing device to capture an image of an area of interest of the skin of the subject, capture an image of the at least one physical collection device, determine the sweat pH of the subject by analyzing the color produced by the physical collection device, and transmit the sweat pH and the image of the area of interest to the server computing system. The server computing system is configured to determine a skincare product recommendation based on the sweat pH; generate a visualization indicating a result of applying the skincare product to the area of interest based on the sweat pH, the image of the area of interest, and the skincare product; and transmit the visualization to the mobile computing device. The instructions also cause the mobile computing device to receive and present the visualization to the subject.

In some embodiments, a method for treating a patient with a skin condition associated with high sweat pH is provided. A sweat pH of skin of the patient is determined by applying at least one physical collection device to the skin of the patient, wherein the physical collection device is configured to produce a color indicative of the sweat pH of the skin; and using a mobile computing device to determine the sweat pH by performing a colorimetric analysis of an image of the at least one physical collection device captured by the mobile computing device. A recommended skincare product to treat the skin condition is determined based on the sweat pH. The recommended skincare product is topically administered to the skin.

In some embodiments, a method of generating a rendering of a face image to indicate a result of a skin treatment regimen is provided. A mobile computing device captures a first image of a physical collection device applied to a skin area of a subject, wherein the physical collection device changes color in response to a pH of sweat excreted by the skin area. The mobile computing device captures a second image of a face of the subject. The mobile computing device determines the pH of the sweat excreted by the skin area based on the first image. The mobile computing device determines a recommended skincare product based on the sweat pH. The mobile computing device determines a predicted face image based on the sweat pH, the recommended skincare product, and the second image. The mobile computing device presents the predicted face image to the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram that illustrates a non-limiting example embodiment of a system according to various aspects of the present disclosure;

FIG. 2 is a block diagram that illustrates various components of a non-limiting example embodiment of a mobile computing device and a non-limiting example embodiment of a server computing system according to various aspects of the present disclosure;

FIGS. 3A and 3B are illustrations showing an example of a first image taken of a subject and a predictive visualization generated of the subject according to various aspects of the present disclosure;

FIGS. 4A-4C are a flowchart that illustrates a non-limiting example embodiment of a method of generating a predictive visualization of a subject's response to a recommended skincare regimen according to various aspects of the present disclosure;

FIG. 5 is a flowchart that illustrates a non-limiting example embodiment of a method of determining pH values based on colors presented by a physical collection device according to various aspects of the present disclosure;

FIG. 6 is a flowchart that illustrates a non-limiting example embodiment of a method of determining pH values based on colors presented by a physical collection device using machine learning according to various aspects of the present disclosure; and

FIG. 7 is a schematic diagram that illustrates a non-limiting example embodiment of a physical collection device according to various aspects of the present disclosure.

### DETAILED DESCRIPTION

Skin pH is a vital component of the normal function of human skin. Dysregulation of the acidity plays a role in a number of diseases, such as atopic dermatitis, eczema, and acne vulgaris. As some non-limiting examples: pH is typically found to be significantly higher in eczematous skin. Higher pH values have been measured in areas corresponding to more intense itching and skin dryness in atopic dermatitis (AD). Free amino acids and urocanic acid, which are involved in creating the acidic milieu of the stratum corneum (SC), are markedly reduced in AD. Filaggrin, a protein precursor of free amino acids, is deficient in AD. Sweat secretion, which is rich in lactic acid that contributes to the acid mantle, is reduced in AD. Impaired barrier function in AD can be explained in part by disturbed synthesis, excretion, and maturation of SC lipids, process that depends on enzymes with acidic pH optima. Aberrant lipid organization, namely increased gel phase relative to the crystalline phase of lamellar structures, has been described in patients with AD. Lamellar liquid crystal formation occurs at pH values of 4.5-6. Colonization with S. aureus is a common feature of patients with AD and is considered a major pathogenic factor in AD. Growth of Staphylococcal strains is maximal at neutral pH and markedly inhibited at pH values around 5. In vitro, P. acnes grows well at pH values between 6 and 6.5 and growth is reduced at pH values less than 6. In a study of acne-prone patients, the number of facial inflammatory lesions was compared in subjects using a conventional alkaline soap versus those using an acidic syndet bar.

Skin care products can either exacerbate skin conditions or ameliorate them. Exposure to exogenous agents such as cleansers, creams, deodorants, and topical antibacterials affect pH and can further exacerbate underlying disease. Selection of topical agents that preserve an acidic environment seems relevant in these patients. Non-soap-based surfactants are known as syndets (synthetic detergent-based bars or liquids). Syndets are generally neutral or acidic, while soap-based cleansers are alkaline. Topical alpha-hydroxy acids (AHA) are common agents used in treating disorders of keratinization. AHA, such as lactic acid, has been shown to increase ceramide production by human keratinocytes by 300% in vitro. Twice daily application of 4% l-lactic acid formulations (pH 3.7-4.0) led to significant improvements in barrier function. Studies have shown beneficial effects of topical acidic electrolyte water (pH 2.0-2.7) on the severity of dermatitis and S. aureus colonization of the skin.

Despite knowledge of these characteristics of how skin conditions react to skin pH, there is not currently any convenient way to collect information regarding pH associated with skin and to use such information to recommend treatment regimens for the skin conditions. What is desired are devices and methods that allow analysis of pH to be used to recommend skincare products.

In some embodiments of the present disclosure, a colorimetric sensor for pH measurement may be used (e.g. litmus paper, microfluidic device with chemistry for colorimetric pH measurement) to collect sweat pH information from a subject. An application is provided on a computing device that detects and analyzes the color change in order to determine the pH of sweat detected by the colorimetric sensor. Based on the determined sweat pH values and questionnaire responses, the application may recommend a skin regimen/treatment to restore skin function.

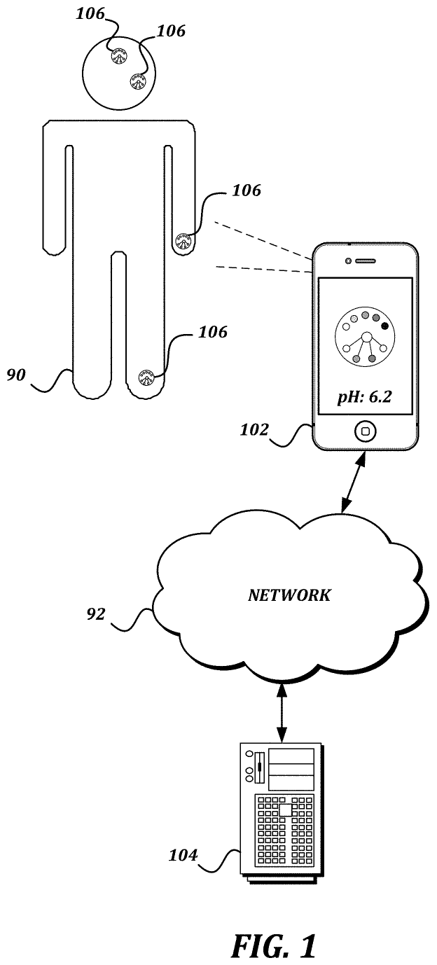

FIG. 1 is a schematic diagram that illustrates a non-limiting example embodiment of a system according to various aspects of the present disclosure. In the illustrated system, the sweat pH of a subject 90 is measured using physical collection devices 106. As shown, four physical collection devices 106 are placed on various skin regions of the subject 90 in order to determine the sweat pH of those regions. In some embodiments, multiple different skin regions may be tested in order to compare the results of those regions to each other, or to build a map of various sweat pH values.

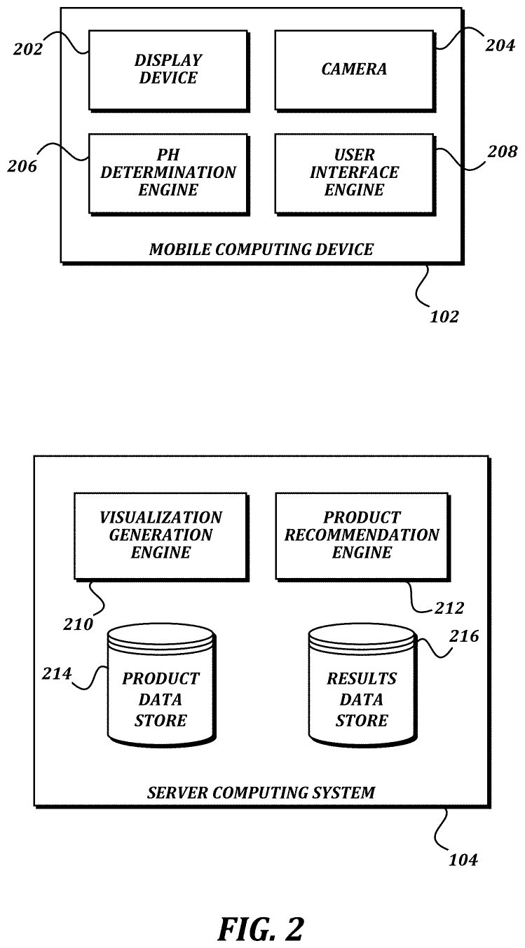

In some embodiments, the physical collection devices 106 may use microfluidics to collect the sweat to be analyzed. In some embodiments, the physical collection devices 106 may change color in response to the detected pH. A mobile computing device 102 is used to determine a sweat pH value detected by each of the physical collection devices 106 based on images of the physical collection devices 106. In some embodiments, the mobile computing device 102 transmits at least the determined sweat pH values to a server computing device 104 via a network 92, and the server computing device 104 may respond with a skincare product recommendation to be presented to the subject 90 by the mobile computing device 102. The network 92 may include any suitable wireless communication technology (including but not limited to Wi-Fi, WiMAX, Bluetooth, 2G, 3G, 4G, 5G, and LTE), wired communication technology (including but not limited to Ethernet, USB, and FireWire), or combinations thereof. FIG. 2 is a block diagram that illustrates various components of a non-limiting example embodiment of a mobile computing device and a non-limiting example embodiment of a server computing system according to various aspects of the present disclosure. The mobile computing device 102 is configured to collect information from a subject 90 that reflects the sweat pH of the subject 90 and/or effectiveness of various skincare products. The mobile computing device 102 then transmits the collected information to the server computing system 104 via the network 92. In some embodiments, the server computing system 104 uses the information to determine a recommendation for a skincare product to be used by the subject 90, and transmits the recommendation back to the mobile computing device 102 to be presented to the subject 90. In some embodiments, the server computing system 104 uses the information to generate visualizations of the subject 90 to show a prediction of how a recommended skincare product would affect a skin condition, and transmits the visualizations back to the mobile computing device 102 to be presented to the subject.

In some embodiments, the mobile computing device 102 may be a smartphone.

In some embodiments, the mobile computing device 102 may be any other type of computing device having the illustrated components, including but not limited to a tablet computing device or a laptop computing device. In some embodiments, the mobile computing device 102 may not be mobile, but may instead be a stationary computing device such as a desktop computing device. In some embodiments, the illustrated components of the mobile computing device 102 may be within a single housing. In some embodiments, the illustrated components of the mobile computing device 102 may be in separate housings that are communicatively coupled through wired or wireless connections (such as a laptop computing device with an external camera connected via a USB cable). The mobile computing device 102 also includes other components that are not illustrated, including but not limited to one or more processors, a non-transitory computer-readable medium, a power source, and one or more network communication interfaces.

As shown, the mobile computing device 102 includes a display device 202, a camera 204, a pH determination engine 206, and a user interface engine 208. In some embodiments, the display device 202 is any suitable type of display device, including but not limited to an LED display, an OLED display, or an LCD display, that is capable of presenting interfaces to the subject 90. In some embodiments, the display device 202 may include an integrated touch-sensitive portion that accepts input from the subject 90. In some embodiments, the camera 204 is any suitable type of digital camera that is used by the mobile computing device 102. In some embodiments, the mobile computing device 102 may include more than one camera 204, such as a front-facing camera and a rear-facing camera.

In some embodiments, the pH determination engine 206 is configured to collect information from the physical collection devices 106 and to determine sweat pH values based on the information. For example, the pH determination engine 206 may use the camera 204 to collect images of the physical collection devices 106, and may then analyze the images to determine the pH represented by colors presented by the physical collection devices 106.

In some embodiments, the user interface engine 208 may be configured to present one or more questionnaires to the subject 90 in order to collect information that may be relevant to the effectiveness of a given skincare product, or to likely environmental effects on a skincare condition. In some embodiments, the user interface engine 208 may be configured to use the camera 204 to capture images of the subject 90, and to present visualizations of the subject 90 received from the server computing system 104.

In some embodiments, the server computing system 104 includes one or more computing devices that each include one or more processors, non-transitory computer-readable media, and network communication interfaces that are collectively configured to provide the components illustrated below. In some embodiments, the one or more computing devices that make up the server computing system 104 may be rack-mount computing devices, desktop computing devices, or computing devices of a cloud computing service.

As shown, the server computing system 104 includes a visualization generation engine 210, a product recommendation engine 212, a product data store 214, and a results data store 216. In some embodiments, the visualization generation engine 210 receives the sweat pH information and an image of the subject 90 from the mobile computing device 102, and uses this information to generate visualizations of the subject 90 over time. The visualizations may include predictions of how a skin condition experienced by the subject 90 will change over time. The predictions may be affected by a recommended skincare product, and/or by questionnaire responses provided by the mobile computing device 102. The visualizations may be transmitted to the mobile computing device 102 to be presented to the subject 90.

In some embodiments, the product recommendation engine 212 receives the sweat pH information and/or the questionnaire information from the mobile computing device 102, and uses the information to determine an appropriate product stored in a product data store 214 that can address a skin condition experienced by the subject 90. In some embodiments, the product recommendation engine 212 provides the recommended products to the mobile computing device 102 to be presented to the subject 90. In some embodiments, the product recommendation engine 212 may also receive feedback from the subject 90 after having used the recommended products, and may store the feedback in a results data store 216 in order to improve future product recommendations.

In general, the word “engine,” as used herein, refers to logic embodied in hardware or software instructions, which can be written in a programming language, such as C, C++, COBOL, JAVA™, PHP, Perl, HTML, CSS, JavaScript, VBScript, ASPX, Microsoft .NET™, Go, and/or the like. An engine may be compiled into executable programs or written in interpreted programming languages. Software engines may be callable from other engines or from themselves. Generally, the engines described herein refer to logical modules that can be merged with other engines, or can be divided into sub-engines. The engines can be stored in any type of computer-readable medium or computer storage device and be stored on and executed by one or more general purpose computers, thus creating a special purpose computer configured to provide the engine or the functionality thereof.

As understood by one of ordinary skill in the art, a “data store” as described herein may be any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

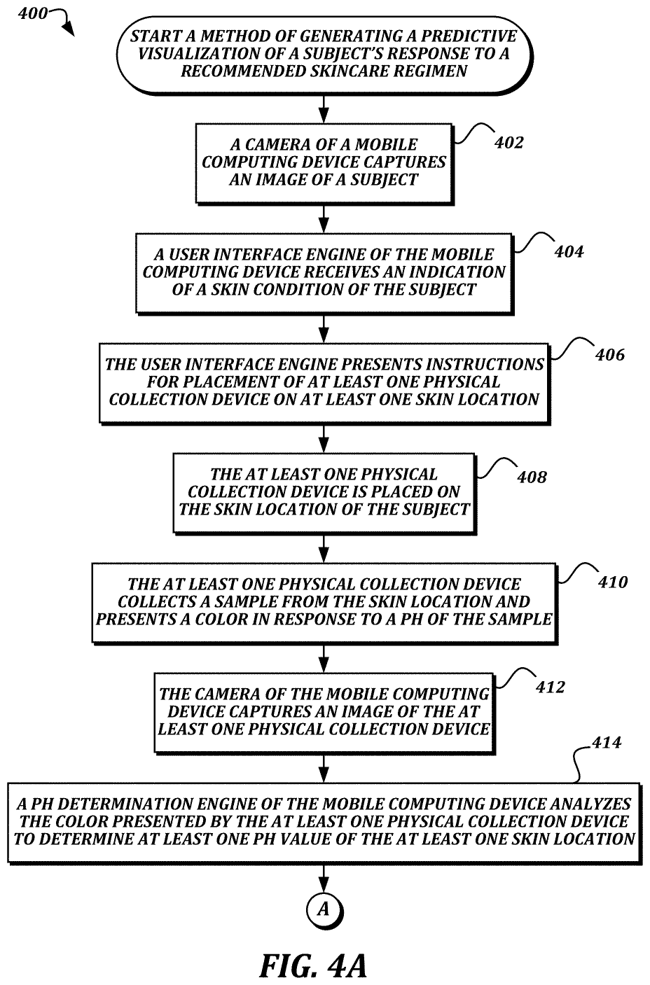

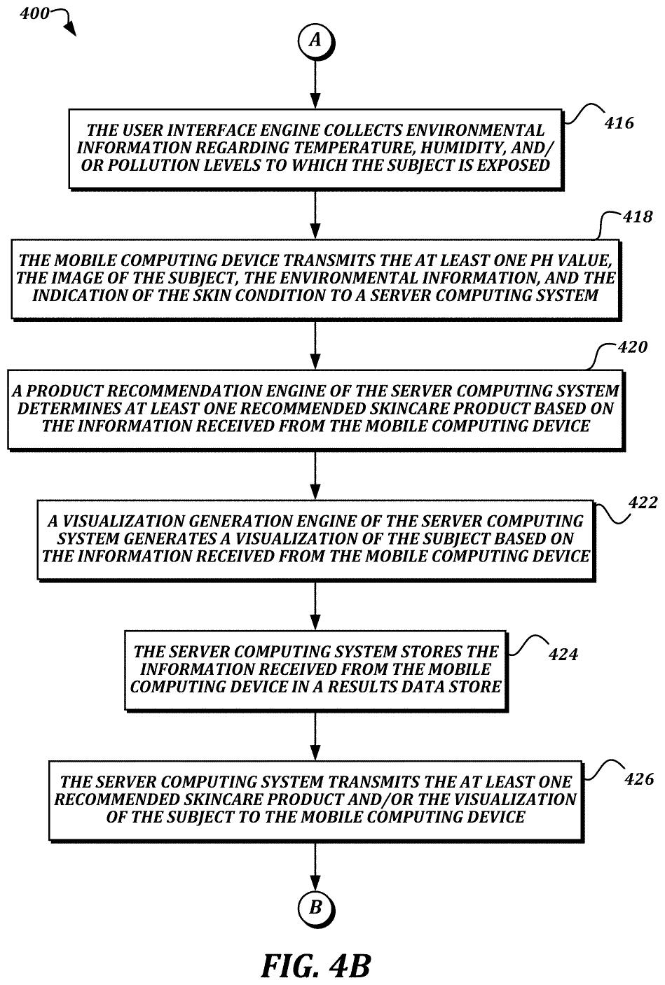

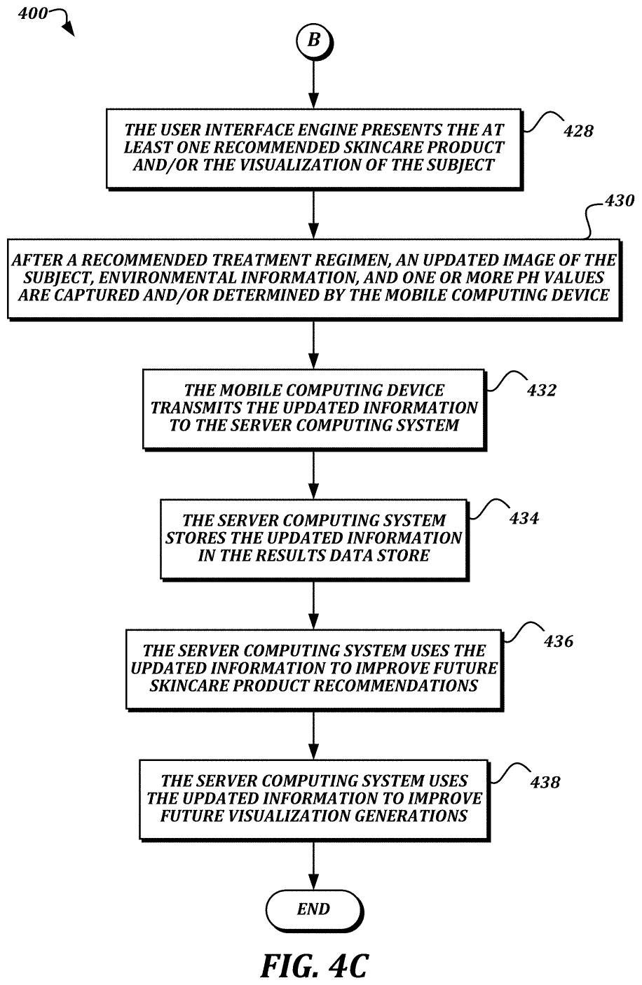

FIGS. 4A-4C are a flowchart that illustrates a non-limiting example embodiment of a method of generating a predictive visualization of a subject's response to a recommended skincare regimen according to various aspects of the present disclosure. In the method 400, the sweat pH of a subject 90 is determined, and an image of the subject is collected. Information regarding the environment of the subject 90 is also collected. A product recommendation is determined, and a visualization is generated to show an expected effect on a skin condition experienced by the subject 90 as a result of using the recommended skincare product.

From a start block, the method 400 proceeds to block 402, where a camera 204 of a mobile computing device 102 captures an image of a subject 90. In some embodiments, the front-facing camera 204 may be used to capture the image of the subject 90, such as a camera 204 that may be used in a “selfie” mode. In other embodiments, a rear-facing camera 204 may be used, particularly in embodiments where an operator other than the subject 90 is operating the mobile computing device 102. At block 404, a user interface engine 208 of the mobile computing device 102 receives an indication of a skin condition of the subject 90. The user interface engine 208 may present a list of conditions, and the subject 90 may select which of the conditions are being experienced (e.g., acne vulgaris, eczema, atopic dermatitis, etc.) from the presented list.

At block 406, the user interface engine 208 presents instructions for placement of at least one physical collection device 106 on at least one skin location. In some embodiments, the instructions may include an image that illustrates where the physical collection device 106 should be placed (e.g., on the forehead, on the cheekbone, on the nose, on the back of the hand, on the top of the foot) in order to collect sweat pH information from a desired area. At block 408, the at least one physical collection device 106 is placed on the skin location of the subject 90 in accordance with the instructions.

At block 410, the at least one physical collection device 106 collects a sample from the skin location and presents a color in response to a pH of the sample. In some embodiments, the at least one physical collection device 106 may include an inlet that is in contact with the skin, and one or more channels that extend from the inlet to one or more reaction cells.

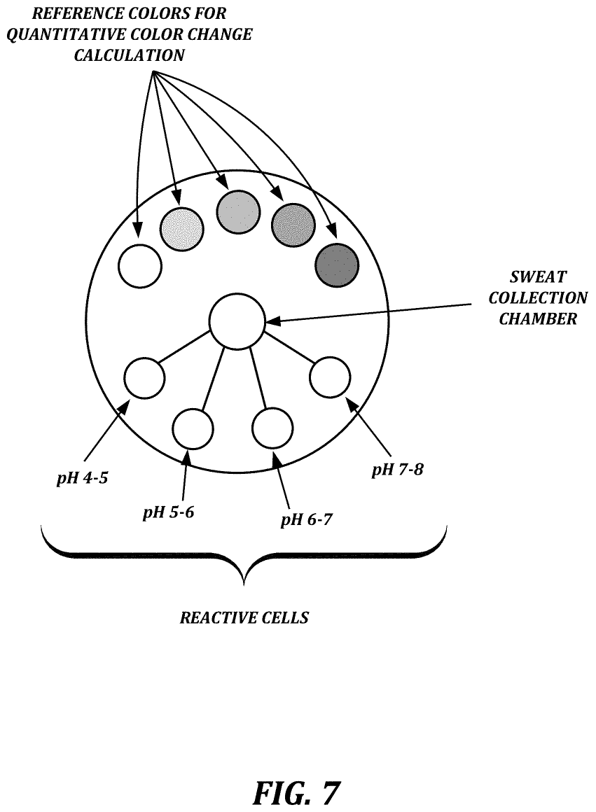

FIG. 7 is a schematic diagram that illustrates a non-limiting example embodiment of a physical collection device according to various aspects of the present disclosure. In FIG. 7, a sweat collection chamber is illustrated in the middle of the device. In the illustrated device, sweat passes from the inlet to the sweat collection chamber, and then to one or more reactive cells. The reactive cells include a substance that changes color based on a detected pH of a liquid placed therein. The colors of the reactive cells can then be compared to the reference colors in order to determine the detected pH. Further details of a non-limiting example of a microfluidic, colorimetric skin pH sensor are described in International Publication No. WO 2018/223044, filed Jun. 1, 2018, by John A. Rogers et al., the entire disclosure of which is hereby incorporated by reference herein.

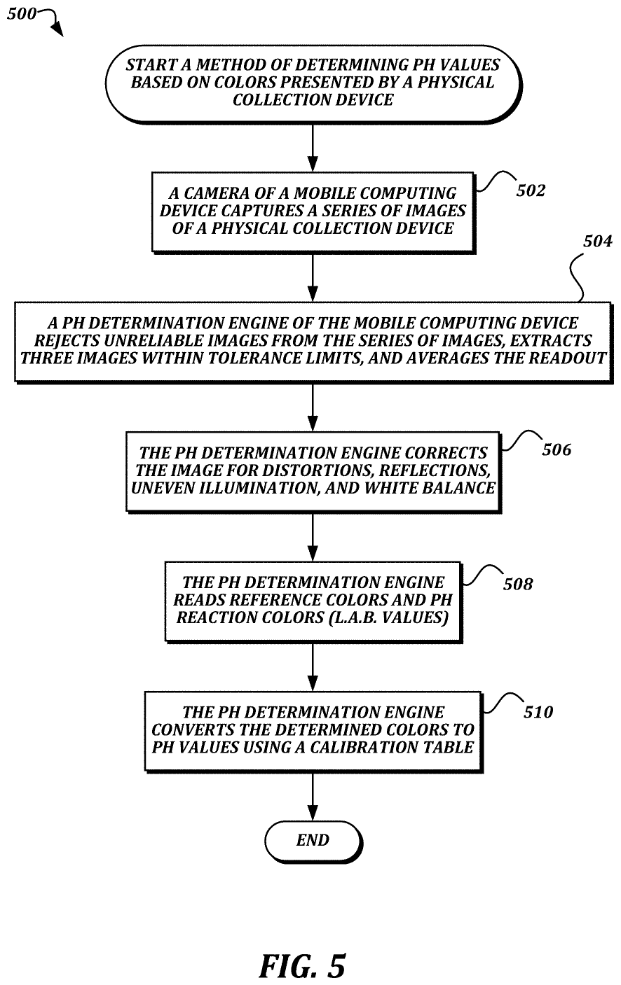

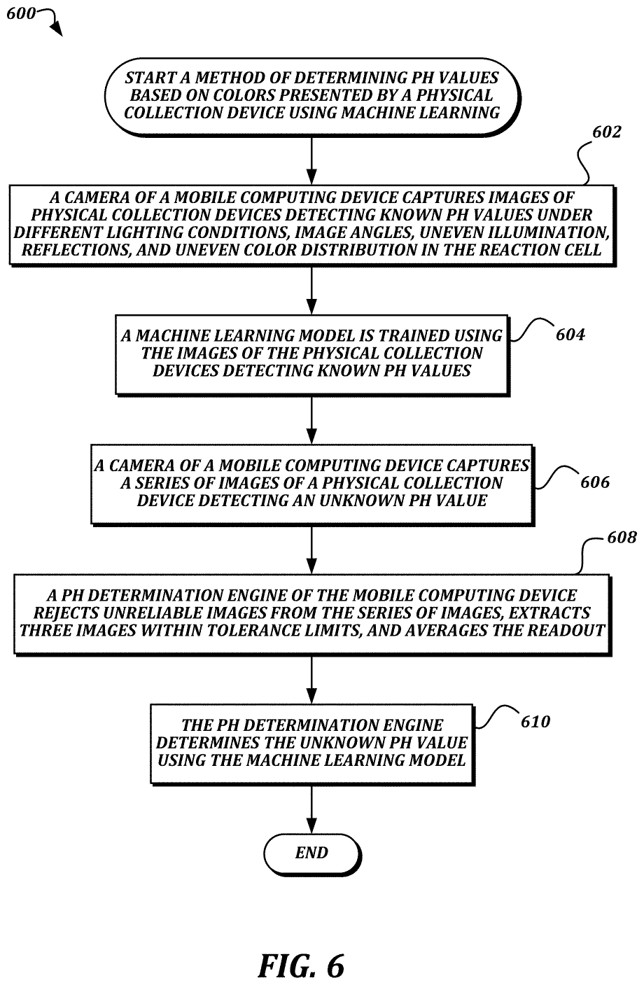

Returning to FIG. 4A, at block 412, the camera 204 of the mobile computing device 102 captures an image of the at least one physical collection device 106. In some embodiments, a rear-facing camera 204 of the mobile computing device 102 may be used to capture the image in order to have the highest possible quality image. At block 414, a pH determination engine 206 of the mobile computing device 102 analyzes the color presented by the at least one physical collection device 106 to determine at least one pH value of the at least one skin location. In some embodiments, the pH determination engine 206 may compare reference colors visible within the image of the physical collection device 106 to correct a white balance of the image and to thereby accurately determine a color of the reactive cells. Further description of methods for determining a pH value based on images of a physical collection device 106 are illustrated in FIGS. 5-6 and are described in detail below. The method 400 then proceeds to a continuation terminal (“terminal A”). From terminal A (FIG. 4B), the method 400 proceeds to block 416, where the user interface engine 208 collects environmental information regarding temperature, humidity, and/or pollution levels to which the subject 90 is exposed. In some embodiments, the user interface engine 208 may present a questionnaire that directly collects the environmental information from the subject 90. In some embodiments, the user interface engine 208 may present a questionnaire that collects information from the subject 90, such as a location, that can be used to look up the environmental information from publicly available information sources for the location.

At block 418, the mobile computing device 102 transmits the at least one pH value, the image of the subject, the environmental information, and the indication of the skin condition to a server computing system 104. At block 420, a product recommendation engine 212 of the server computing system 104 determines at least one recommended skincare product based on the information received from the mobile computing device 102. In some embodiments, the product recommendation engine 212 may retrieve at least one recommended skincare product from the product data store 214 that is specifically associated with regulating the detected pH in order to treat the skin condition subject to the specified environmental conditions. In some embodiments, a machine learning model such as a recommender system may be used to determine the at least one recommended skincare product.

At block 422, a visualization generation engine 210 of the server computing system 104 generates a visualization of the subject 90 based on the information received from the mobile computing device 102. In some embodiments, a prediction of an effect that the recommended skincare product will have on the skin condition subject to the specified environmental conditions will be generated. For example, the visualization generation engine 210 may determine that a given skincare product will adjust a sweat pH from a problematic value detected by the system to a normal value, and may then use computer image generation techniques generate a visualization that depicts the subject 90 as having less evidence of the skin condition.

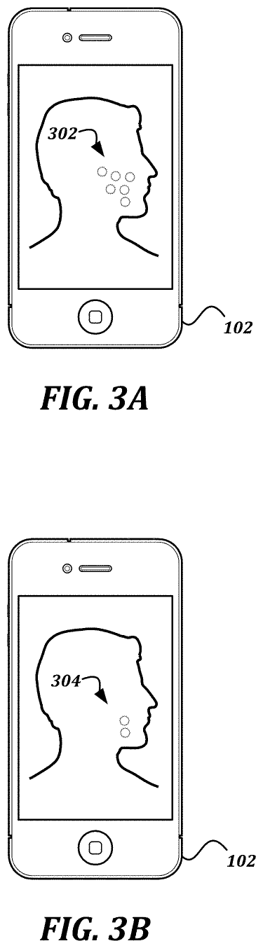

FIGS. 3A and 3B are illustrations showing an example of a first image taken of a subject and a predictive visualization generated of the subject according to various aspects of the present disclosure. In FIG. 3A, an image captured of the subject is presented, showing multiple acne lesions. In FIG. 3B, a predictive visualization of the subject is presented, showing a predicted reduction in acne lesions after recommended use of a recommended skincare product.

Returning to FIG. 4B, at block 424, the server computing system 104 stores the information received from the mobile computing device 102 in a results data store 216. In some embodiments, this information may later be associated with feedback received from the subject 90, and/or to improve recommendations provided by the product recommendation engine 212. At block 426, the server computing system 104 transmits the at least one recommended skincare product and/or the visualization of the subject 90 to the mobile computing device 102.

The method 400 then proceeds to another continuation terminal (“terminal B”). From terminal B (FIG. 4C), the method 400 proceeds to block 428, where the user interface engine 208 presents the at least one recommended skincare product and/or the visualization of the subject 90. At block 430, after a recommended treatment regimen, an updated image of the subject 90, environmental information, and one or more pH values are captured and/or determined by the mobile computing device 102. In some embodiments, similar techniques as those described in blocks 406-416 may be used to again capture this information. At block 432, the mobile computing device 102 transmits the updated information to the server computing system 104. At block 434, the server computing system 104 stores the updated information in the results data store 216. In some embodiments, the updated information is associated with the original information stored at block 424.

At block 436, the server computing system 104 uses the updated information to improve future skincare product recommendations, and at block 438, the server computing system 104 uses the updated information to improve future visualization generations. For example, the server computing system 104 may compare the updated image of the subject 90 to the original image collected of the subject, and may use computer vision techniques such as convolutional neural networks to detect skin regions that exhibit signs of the skin condition. The continued presence, absence, or reduction of signs of the skin condition can be used to determine the effectiveness of the recommended skincare product, and this effectiveness can then be used to either increase or reduce the likelihood that the skincare product will be recommended in the future for subjects that share traits (such as similar sweat pH or similar environmental factors) with the subject 90. The differences between the updated image and the original image can also be used to generate similar differences in visualizations for other subjects.

The method 400 then proceeds to an end block and terminates.

FIG. 5 is a flowchart that illustrates a non-limiting example embodiment of a method of determining pH values based on colors presented by a physical collection device according to various aspects of the present disclosure.

From a start block, the method 500 proceeds to block 502, where a camera 204 of a mobile computing device 102 captures a series of images of a physical collection device 106. In some embodiments, multiple snapshots of the physical collection device 106 may be separately captured. In some embodiments, a video of the physical collection device 106 may be captured, and the series of images may be extracted from the video. In some embodiments, the images of the series of images may be captured from multiple angles and/or under different lighting conditions.

At block 504, a pH determination engine 206 of the mobile computing device 102 rejects unreliable images from the series of images, extracts three images within tolerance limits, and averages the readout. In some embodiments, a convolutional neural network or other machine learning model may be used to extract features from the images of the series of images, and images that are found to be lacking expected features may be rejected as unreliable. In some embodiments, the pH determination engine 206 may check to make sure that the images have a brightness level, a white level, or other value within an acceptable tolerance range, and may reject images that are not within the acceptable tolerance range.

At block 506, the pH determination engine 206 corrects the image for distortions, reflections, uneven illumination, and white balance. In some embodiments, detected reflections may be excised from the image. In some embodiments, a perspective/viewpoint of the camera 204 may be determined based on a detected shape of the physical collection device 106 in the image, and the image may be warped by the pH determination engine 206 to reproduce the expected shape of the physical collection device 106. In some embodiments, reference colors visible on the physical collection device 106 or otherwise within the image (such as a color reference card) may be used for correcting the uneven illumination and/or white balance.

At block 508, the pH determination engine 206 reads reference colors and pH reaction colors (L.A.B. values). In some embodiments, a color space other than L.A.B., such as RGB, HSV, HSL, YPbPr, CMYK, YUV, or TSL may be used. At block 510, the pH determination engine 206 converts the determined colors to pH values using a calibration table. In some embodiments, the calibration table correlates determined colors to specific pH values and/or ranges.

The method 500 then proceeds to an end block and terminates.

FIG. 6 is a flowchart that illustrates a non-limiting example embodiment of a method of determining pH values based on colors presented by a physical collection device using machine learning according to various aspects of the present disclosure.

From a start block, the method 600 proceeds to block 602, where a camera 204 of a mobile computing device 102 captures images of physical collection devices 106 detecting known pH values under different lighting conditions, image angles, uneven illumination, reflections, and uneven color distribution in the reaction cell. In some embodiments, the physical collection devices 106 may be exposed to samples of solution of having known pH values. For example, a solution having a pH value of 6.5 may be prepared, and a physical collection device 106 may be exposed to this solution. As another example, a sweat pH reading may be taken using a sweat pH probe or other technique for determining a ground truth sweat pH reading, and a physical collection device 106 may then be used to obtain a reading from the same area of skin.

At block 604, a machine learning model is trained using the images of the physical collection devices 106 detecting known pH values. In some embodiments, the known pH values are used to tag the images of the physical collection devices 106 to create a set of supervised training data, and a machine learning model such as an artificial neural network may be trained with the training data using any suitable technique, including but not limited to gradient descent. The resulting machine learning model will accept an image of a physical collection device 106 as input, and will output a detected pH value that is represented by the physical collection device 106.

At block 606, a camera of a mobile computing device 102 captures a series of images of a physical collection device 106 detecting an unknown pH value. At block 608, a pH determination engine 206 of the mobile computing device 102 rejects unreliable images from the series of images, extracts three images within tolerance limits, and averages the readout. The actions at this step are similar to the actions described above in block 504, and so are not described again here for the sake of brevity.

At block 610, the pH determination engine 206 determines the unknown pH value using the machine learning model. In some embodiments, the average readouts of the three extracted images may be provided as input to the machine learning model, and the machine learning model may output the pH value. In some embodiments, the normalization steps of averaging the readouts may be skipped, and the raw images may instead be provided to the machine learning model for analysis.

The method 600 then proceeds to an end block and terminates.

While illustrative embodiments have been illustrated and described, it will be appreciated that various changes can be made therein without departing from the spirit and scope of the invention.

The embodiments of the invention in which an exclusive property or privilege is claimed are defined as follows:

1. A system for generating predictive visualizations of a result of a skin treatment regimen, the system comprising:
a server computing system comprising one or more computing devices;
at least one physical collection device configured to produce a color indicative of a sweat pH of a subject on whose skin the physical collection device is placed; and
a mobile computing device comprising:
at least one processor;
a display device;
a camera; and
a non-transitory computer-readable medium having computer-executable instructions stored thereon that, in response to execution by the at least one processor, cause the mobile computing device to:
capture an image of an area of interest of the skin of the subject;
capture an image of the at least one physical collection device;
determine the sweat pH of the subject by analyzing the color produced by the physical collection device; and
transmit the sweat pH and the image of the area of interest to the server computing system;
wherein the server computing system is configured to:
determine a skincare product recommendation based on the sweat pH;
generate a visualization indicating a result of applying the skincare product to the area of interest based on the sweat pH, the image of the area of interest, and the skincare product; and
transmit the visualization to the mobile computing device;
wherein the instructions further cause the mobile computing device to:
receive and present the visualization to the subject.

2. The system of claim 1, wherein the instructions further cause the mobile computing device to:
collect environmental information regarding at least one of temperature, humidity, and pollution levels; and
transmit the environmental information to the server computing system;
wherein the determination of the skincare product recommendation is further based on the environmental information.

3. The system of claim 1, wherein the instructions further cause the mobile computing device to:
collect information regarding a medical condition experienced by the subject; and
transmit the information regarding the medical condition to the server computing system;
wherein the determination of the skincare product recommendation is further based on the information regarding the medical condition.

4. The system of claim 3, wherein the medical condition is selected from a group consisting of eczema, atopic dermatitis, and acne vulgaris.

5. The system of claim 3, wherein the visualization illustrates a predicted improvement in the medical condition based on a predicted effect of application of the skincare product.

6. The system of claim 1, wherein the visualization illustrates a predicted pH trend caused by application of the skincare product.

7. The system of claim 1, wherein the at least one physical collection device includes a plurality of physical collection devices to be placed on different skin regions, and wherein the recommendation includes at least two skincare products recommended for use on different skin regions.

8. The system of claim 7, wherein the instructions further cause the mobile device to present instructions for placement of each physical collection device.

9. The system of claim 1, wherein the at least one physical collection device includes a microfluidic patch.

10. A method for treating a patient with a skin condition associated with high sweat pH, the method comprising:
determining a sweat pH of skin of the patient by:
applying at least one physical collection device to the skin of the patient, wherein the physical collection device is configured to produce a color indicative of the sweat pH of the skin; and
using a mobile computing device to determine the sweat pH by performing a colorimetric analysis of an image of the at least one physical collection device captured by the mobile computing device;
determining a recommended skincare product to treat the skin condition based on the sweat pH; and
topically administering the recommended skincare product to the skin.

11. The method of claim 10, wherein the skin condition is selected from a group consisting of atopic dermatitis, eczema, and acne vulgaris.

12. The method of claim 10, wherein the recommended skincare product is selected from a group consisting of a syndet, a soap-based cleanser, an alpha-hydroxy acid, and an acidic electrolyte water.

13. The method of claim 10, wherein the at least one physical collection device is a microfluidic patch.

14. The method of claim 10, further comprising collecting environmental information regarding at least one of temperature, humidity, and pollution levels, wherein the determination of the recommended skincare product is further based on the environmental information.

15. A method of generating a rendering of a face image to indicate a result of a skin treatment regimen, the method comprising:
capturing, by a mobile computing device, a first image of a physical collection device applied to a skin area of a subject, wherein the physical collection device changes color in response to a pH of sweat excreted by the skin area;
capturing, by the mobile computing device, a second image of a face of the subject;
determining, by the mobile computing device, the pH of the sweat excreted by the skin area based on the first image;
determining, by the mobile computing device, a recommended skincare product based on the sweat pH;
determining, by the mobile computing device, a predicted face image based on the sweat pH, the recommended skincare product, and the second image; and
presenting, by the mobile computing device, the predicted face image to the subject.

16. The method of claim 15, further comprising collecting, by the mobile computing device, environmental information regarding at least one of temperature, humidity, and pollution levels;
wherein the determination of the recommended skinc are product and the determination of the predicted face image are both further based on the environmental information.

17. The method of claim 15, further comprising receiving, by the mobile computing device, an indication of a medical condition experienced by the subject;
wherein determining the predicted face image is further based on the indication of the medical condition, and the predicted face image indicates an effect of the recommended skincare product on the medical condition.

18. The method of claim 17, wherein the medical condition is selected from a group consisting of eczema, atopic dermatitis, and acne vulgaris.
